(19) United States
(12) Patent Application Publication
(10) Pub. No.: US 2019/0002385 A1
(43) Pub. Date: Jan. 3, 2019

(54) COMPOSITIONS FOR IMPROVED PRODUCTION OF ACRYLIC ACID

(71) Applicant: Novomer, Inc., Waltham, MA (US)

(72) Inventors: Sadesh H. Sookraj, Cambridge, MA (US); Kyle Evan Sherry, Rochester, NY (US)

(73) Assignee: Novomer, Inc.

(21) Appl. No.: 15/640,324

(22) Filed: Jun. 30, 2017

(51) Int. Cl. C07C 51/09; C08J 11/12

(52) U.S. Cl. CPC C07C 51/09; C08J 2367/04; C08J 11/12

(57) ABSTRACT

The present invention is directed to compositions which may undergo thermolysis to produce a higher purity acrylic acid product. In preferred embodiments of the present invention, the compositions comprise polypropiolactone and one or more active salts. The one or more active salts may catalyze thermolysis of the polypropiolactone so that the polymer depolymerizes into acrylic acid monomers. Certain concentrations of the one or more active salts result in higher purity acrylic acid products of thermolysis. In certain preferred embodiments, the one or more active salts include an acrylate group which may decompose under thermolysis to provide acrylic acid and thus decrease the concentration of undesirable contaminants in the acrylic acid product. In certain preferred embodiment, the one or more active salts comprise sodium acrylate.

### FIELD OF THE INVENTION

This invention generally relates to compositions for the improved production of acrylic acid through a thermolysis reaction. Specifically, embodiments of the present invention include compositions comprising polypropiolactone and one or more active salt which may catalyze the thermolysis of polypropiolactone to produce acrylic acid. Advantageously, embodiments of the present invention may be more efficiently transported and stored and may provide higher purity acrylic acid products of thermolysis.

### BACKGROUND OF THE INVENTION

Polypropiolactone, termed “PPL” for the purposes of this application, is a biodegradable polymer that can be useful material in many manufacturing and industrial applications. The physical and chemical characteristics of PPL provide for safer transportation and storage over extended periods of time with decreased quality concerns. PPL is also a useful precursor because the polymer may undergo a chemical process known as thermolysis to produce acrylic acid.

Generally, thermolysis is a chemical decomposition reaction caused by heat. Thermolysis of PPL may proceed by two known reactions. In one reaction, a PPL polymer with a chain length equal to (n) decomposes into a PPL polymer with a chain length (n−1) and a molecule of acrylic acid. In another reaction, a PPL polymer with a chain length (n) decomposes into a PPL polymer with a chain length (n−x) and a PPL polymer with a chain length (x), where (x) is greater than or equal to 2.

Under certain reaction conditions, acrylic acid may be susceptible to auto-polymerization. In one auto-polymerization reaction, a first molecule of acrylic acid is added to a second molecule of acrylic acid to form a di-acrylic acid ester, which is identical to a PPL polymer with a chain length of 2. There is no known inhibitor which will prevent the addition of one molecule of acrylic acid to another. However, the di-acrylic acid ester may readily undergo thermolysis and decompose back into two molecules of acrylic acid. In a second auto-polymerization reaction, multiple molecules of acrylic acid undergo radical polymerization to form larger chains of polyacrylic acid. These larger chains of polyacrylic acid are not likely to convert back into individual molecules of acrylic acid under thermolysis conditions.

Radical polymerization of acrylic acid may be limited with the use of certain known inhibitors. However, these radical polymerization inhibitors may be costly, inefficient, and/or difficult to source. Additionally, conventional thermolysis reactor systems may not efficiently thermolyze polypropiolactone or may not effectively produce acrylic acid vapor effluent. There exists a need for compositions for an intermediate which may be thermolyzed to produce higher purity acrylic acid products. The present invention satisfies this need by providing compositions comprising PPL and one or more active salt.

### SUMMARY OF THE INVENTION

The present invention is directed to compositions comprising PPL and one or more active salt. Advantageously, embodiments of the present invention may be more easily transported and stored with decreased safety concerns and may provide higher purity acrylic acid products of thermolysis.

In preferred embodiments of the present invention, the compositions comprising PPL and one or more active salt may be a stable material that can be safely transported and stored for extended periods without the safety concerns or the quality declines attendant with shipping and storing acrylic acid. If acrylic acid is needed, then the compositions of the present invention may be readily decomposed in a thermolysis reaction vessel to produce higher purity acrylic acid. Therefore, certain embodiments the present invention enable access to acrylic acid in a safer and/or less expensive and/or highly configurable manner. In certain embodiments, the liberated acrylic acid is of a purity suitable for direct use in the manufacture of acrylic acid polymers such as SAPs.

In certain preferred embodiments, the compositions may comprise PPL as a liquid and/or solid and the PPL may have a varying chain length. In certain preferred embodiments, the PPL preferably may be present at a high concentration by weight. In some embodiments, the compositions may also include β-propiolactone and/or sodium acrylate. The β-propiolactone (“BPL”) preferably may be present in the compositions at a lower concentration by weight. The sodium acrylate preferably may be present in the compositions at a lower concentration by weight.

While this disclosure is susceptible to various modifications and alternative forms, specific exemplary embodiments thereof have been shown by way of example in the drawings and have herein been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

### BRIEF DESCRIPTION OF THE DRAWINGS

The present invention can be better understood by reading the following detailed description of certain preferred embodiments, reference being made to the accompanying drawings in which:

FIG. 1 illustrates a H NMR graph of an acrylic acid product formed from thermolysis of a composition of the present invention comprising one or more active salt having a concentration between 0.01% and 1% by weight.

FIG. 2 illustrates a H NMR graph of an acrylic acid product formed from thermolysis of a composition of the present invention comprising one or more active salt having a concentration between 1% and 5% by weight.

FIG. 3 illustrates a H NMR graph of an acrylic acid product formed from thermolysis of a composition of the present invention comprising one or more active salt having a concentration between 5% and 10% by weight.

### DETAILED DESCRIPTION OF EMBODIMENTS

The present invention is directed to novel compositions which may undergo thermolysis to produce higher purity acrylic acid. The compositions of the present invention are comprised of PPL and one or more active salts. In certain embodiments, thermolysis may decompose the PPL to produce acrylic acid at a temperature greater than 100° C., greater than 150° C., greater than 175° C. greater than 200° C., or greater than about 220° C.

In preferred embodiments, the present invention is directed to compositions comprising PPL at a concentration at least 90% by weight. More preferably, compositions of the present invention comprise PPL at a concentration of at least 95% by weight. Most preferably, compositions of the present invention comprise PPL at a concentration of at least 98% by weight. The compositions of the present invention preferably include PPL at a concentration less than 100% by weight.

In certain embodiments, the PPL may be characterized as a liquid. In certain embodiments, such liquid PPL compositions have a higher percentage of relatively low-molecular weight polymer chains. In certain embodiments, the number average molecular weight (MN) of the PPL produced is between about 200 g/mol and about 10,000 g/mol. In certain embodiments, the MN of the PPL produced is less than about 5,000 g/mol, less than about 3,000 g/mol, less than about 2,500 g/mol, less than about 2,000 g/mol, less than about 1,500 g/mol, less than about 1,000 g/mol, or less than about 750 g/mol. In certain embodiments, the PPL produced comprises oligomers containing from 2 to about 10 monomer units. In certain embodiments, such oligomers comprise cyclic oligomers. In certain embodiments, cyclic oligomers contain, on average about 2 monomer units, about 3 monomer units, about 4 monomer units, about 5 monomer units, about 6 monomer units, up to about 10 monomer units, or mixtures of two or more of these materials.

In certain embodiments, the PPL may be characterized as a solid. In certain embodiments, solid PPL compositions comprise a higher percentage of high molecular weight polymer chains. In certain embodiments, such high molecular PPL is characterized in that it has an M between about 10,000 g/mol and about 1,000,000 g/mol. In certain embodiments, high molecular PPL is characterized in that it has an M greater than about 10,000 g/mol, greater than about 20,000 g/mol, greater than about 50,000 g/mol, greater than about 70,000 g/mol, greater than about 100,000 g/mol, greater than about 150,000 g/mol, greater than about 200,000 g/mol, or greater than about 300,000 g/mol.

In preferred embodiments, the formation of the PPL includes carbonylation of ethylene oxide with carbon monoxide and a carbonylation catalyst to provide BPL which is then polymerized to provide PPL. In certain preferred embodiments, the BPL is not isolated from one reactor and polymerized in a second reactor, but rather is carbonylated and polymerized in situ to provide the PPL. In certain preferred embodiments, the BPL may be polymerized using an active salt as a catalyst. Advantageously, the novel compositions of the present invention may include residual active salt polymerization catalysts which are also thermolysis catalysts.

Polymerization of BPL to form PPL may be performed with various active salts for polymerization initiation including but not limited to alcohols, amines, polyols, polyamines, diols, metals (e.g., lithium, sodium, potassium, magnesium, calcium, zinc, aluminum, titanium, cobalt, etc.) metal oxides, carbonates of alkali- and alkaline earth metals, borates, and silicates. The polymerization process includes covalently incorporating such active salt polymerization initiators into a polymer chain. In certain embodiments, the present invention provides a solution to a potentially undesirable effect of this covalently bound initiator: namely, when the PPL is depolymerized to provide acrylic acid, the active salt polymerization initiator may also be liberated and may act as a contaminant in the acrylic acid produced. Therefore, in certain preferred embodiments, the step of polymerizing the BPL comprises contacting the BPL with a polymerization catalyst comprising an active salt including an acrylate. Polymers formed using an active salt including an acrylate as polymerization initiators have the added advantage that fewer non-acrylate materials arising from the bound initiator will contaminate the subsequent acrylic acid stream produced from thermolysis of the polymer. In certain preferred embodiments, the active salt comprises sodium acrylate and/or potassium acrylate.

In preferred embodiments, the present invention is directed to compositions comprising one or more active salt at a concentration of at least 0.01% by weight. More preferably, compositions of the present invention comprise one or more active salt at a concentration of at least 0.1% by weight. Most preferably, compositions of the present invention comprise one or more active salt at a concentration of at least 1% by weight. The compositions of the present invention preferably include one or more active salt at a concentration of less than 10% by weight.

Preferably, the one or more active salt comprises an alkali salt such as sodium carbonate and potassium carbonate. More preferably, the one or more active salt may be an acrylate salt such as sodium acrylate and potassium acrylate. Most preferably, the one or more active salt is sodium acrylate. In at least one embodiment, the one or more active salt comprises tert-butyl ammonium acrylate.

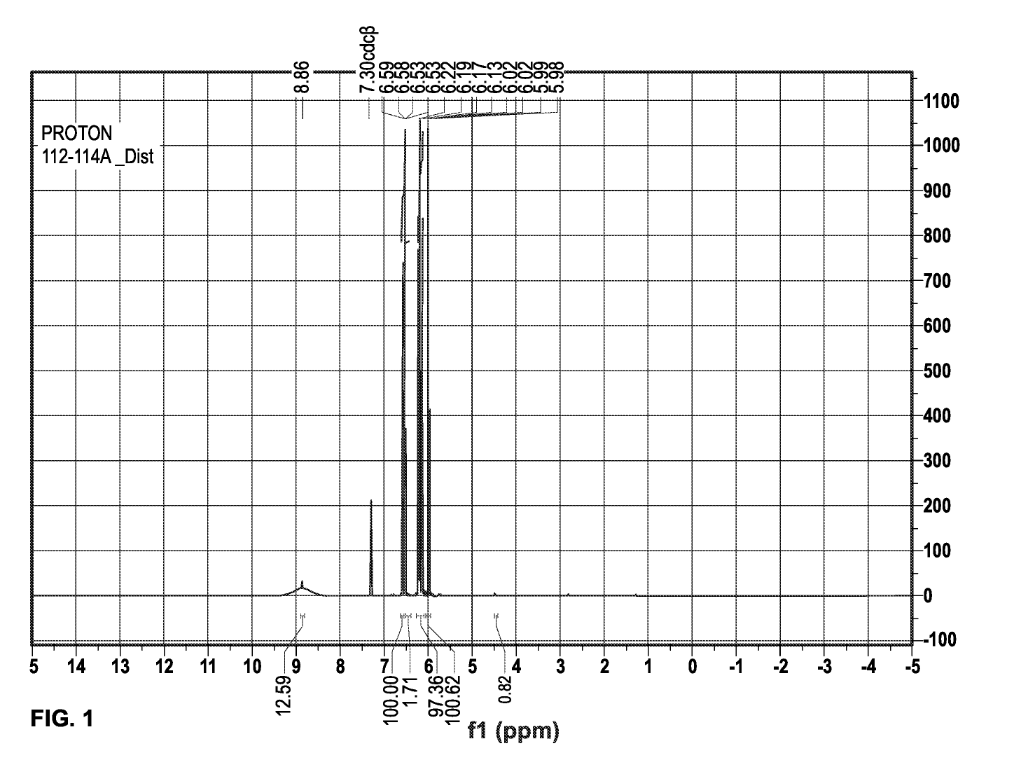

FIG. 1 illustrates the hydrogen nuclear magnetic resonance (“H NMR”) graph of an acrylic acid product produced through thermolysis of a composition of the present invention comprising one or more active salt with a concentration between 0.01% and 1% by weight.

The acrylic acid product represented by the FIG. 1 illustration is produced using a lab-scale batch thermolysis process vessel comprising a two-necked round-bottom glass flask of 25 mL approximate internal volume. The thermolysis process vessel is equipped with an internal thermocouple and the top center opening is equipped with a separation chamber comprising a Vigreuxt™ column oriented coaxially (similar to Ace Glass™ item #6578-04), followed by an adapter with an additional thermocouple to monitor vapor temperature, followed by a water-cooled condenser, and finally a four-armed product receiver in a dry ice/acetone-cooled dewar. The thermolysis process vessel includes a heater comprising a fabric heating mantle, the power to which is controlled by a temperature controller that receives feedback from the thermocouple inside the thermolysis process vessel. The thermolysis process vessel includes a stirrer comprising a magnetic stir plate and a PTFE-coated stir bar.

The feed stream introduced to the thermolysis process vessel is comprised of 5 mg phenothiazine and 6.660 g of PPL produced from ring-opening polymerization of solvent-free BPL in the presence of sodium acrylate at a concentration of 1 mol per 6,000 mol of BPL and phenothiazine at a concentration of 200 ppmw in BPL. The feed stream is heated in the thermolysis process vessel to 90° C. to melt and begin stirring. The thermolysis process vessel is brought under vacuum to an absolute pressure of approximately 400 torr, and the thermolysis process vessel temperature setpoint was set to 230° C. Internal reflux was observed inside the reaction flask within minutes.

The product sample 112-114A_Dist had a mass of 0.516 g, of a total 5.667 g total distillate collected. The HNMR analysis suggests an average acrylic acid content in 112-114A_Dist of 99.2%. The balance consists of di-acrylic acid ester and traces of other PPL oligomers where n>2.

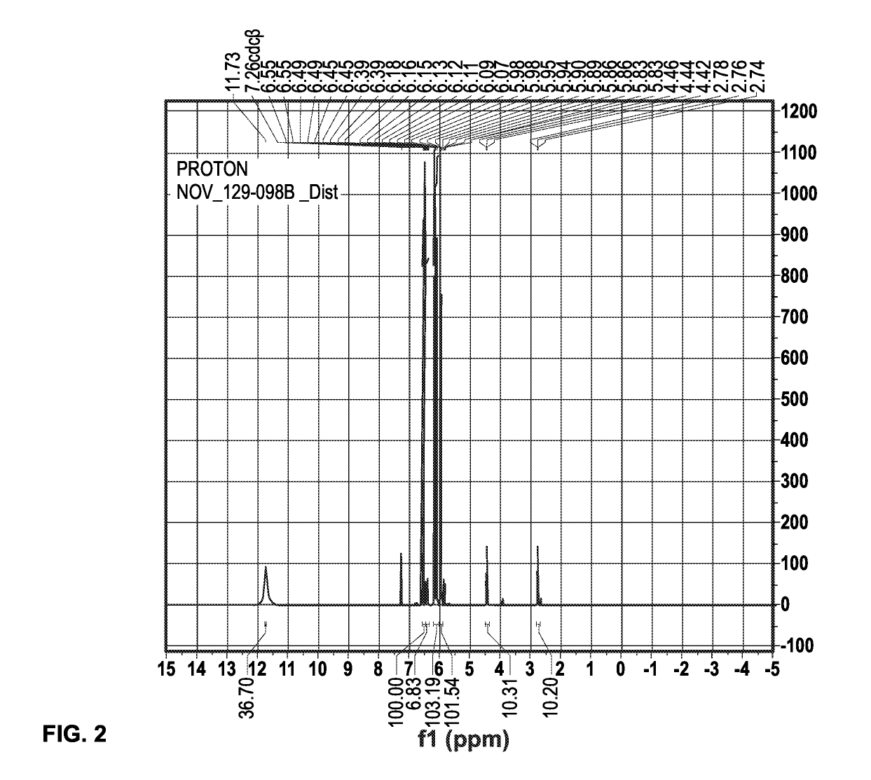

FIG. 2 illustrates the hydrogen nuclear magnetic resonance graph of an acrylic acid product produced from thermolysis of a composition of the present invention comprising one or more active salt having a concentration between 1% and 5% by weight.

The acrylic acid product represented by the FIG. 2 illustration is produced using a lab-scale batch thermolysis process vessel comprising a two-necked round-bottom glass flask of 25 mL approximate internal volume. The thermolysis process vessel is equipped with an internal thermocouple and the top center opening of the thermolysis process vessel includes a separation chamber comprising a short-path distillation apparatus including a short path still (similar to Ace Glass™ item #6554-06) with an additional thermocouple to monitor vapor temperature, followed by a water-cooled condenser, and finally a four-armed product receiver in a dry ice/acetone-cooled dewar. The thermolysis process vessel includes a heater comprising a fabric heating mantle, the power to which is controlled by a temperature controller that receives feedback from the thermocouple inside the thermolysis process vessel. The thermolysis process vessel includes a stirrer comprising a magnetic stir plate and a PTFE-coated stir bar.

The feed stream introduced to the thermolysis process vessel comprises 90 mg dry sodium acrylate, 5 mg phenothiazine, and 4.995 g of PPL produced from ring-opening polymerization of solvent-free BPL in the presence of sodium acrylate at a concentration of 1 mol per 6,000 mol of BPL and phenothiazine at a concentration of 200 ppmw in BPL. The feed stream in the thermolysis process vessel is heated to 90° C. to melt and begin stirring. The thermolysis process vessel is brought under vacuum to an absolute pressure of approximately 700 torr, and the thermolysis process vessel temperature setpoint is set to 210° C. Internal reflux is observed inside the thermolysis process vessel within minutes and the thermolysis process vessel is held at 210° C. for 10 minutes.

The product sample 129-098B_Dist HNMR analysis suggests an average acrylic acid content in 129-098B_Dist of 90.7% by mass. The balance consists of di-acrylic acid ester and traces of other PPL oligomers where n>2.

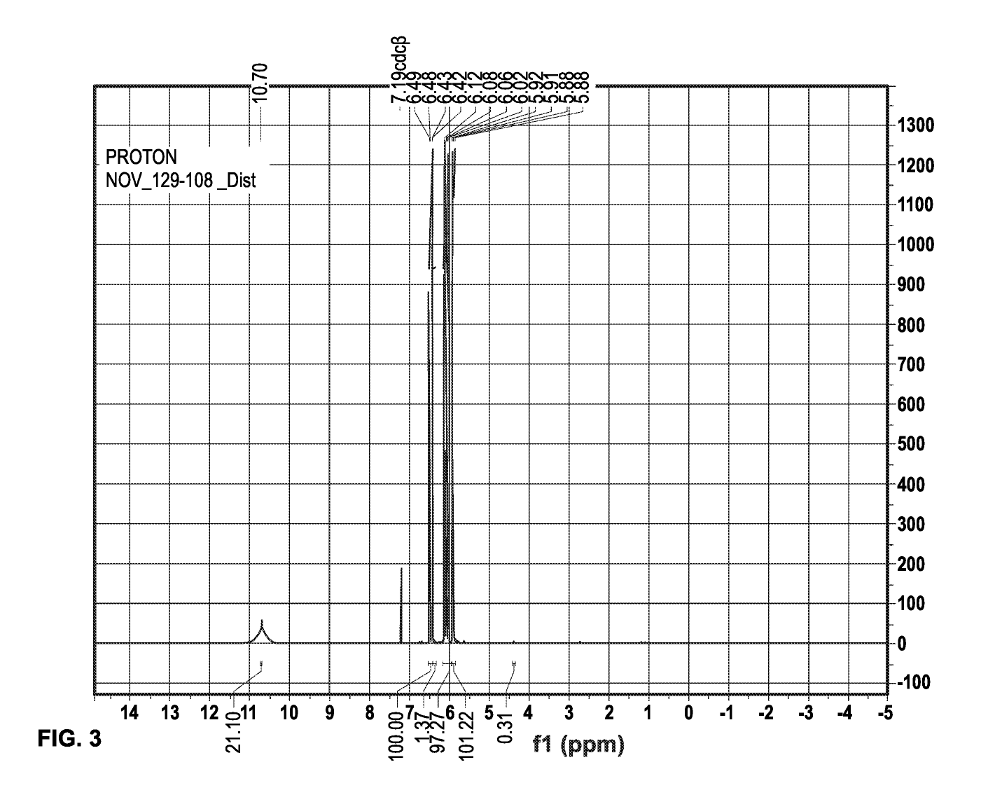

FIG. 3 illustrates the hydrogen nuclear magnetic resonance graph of an acrylic acid product produced from thermolysis of a composition of the present invention comprising one or more active salt having a concentration between 5% and 10% by weight.

The acrylic acid product represented by the FIG. 3 illustration was produced using a lab-scale batch thermolysis process vessel comprising a two-necked round-bottom glass flask of 50 mL approximate internal volume. The thermolysis process vessel includes an internal thermocouple and a separation chamber located at the top center opening in the thermolysis process vessel. The separation chamber comprises a distillation apparatus including two Vigreux™ columns in series oriented coaxially (each similar to Ace Glass™ item #6578-04), followed by an adapter with an additional thermocouple to monitor vapor temperature, followed by a water-cooled condenser, and finally a 50 mL round-bottom product receiver in a dry ice/acetone-cooled dewar. The thermolysis process vessel includes a heater comprising a fabric heating mantle, the power to which is controlled by a temperature controller that receives feedback from the thermocouple inside the thermolysis process vessel. The thermolysis process vessel includes a stirrer comprising a magnetic stir plate and a PTFE-coated stir bar.

The feed stream introduced to the thermolysis process vessel comprises 1000 mg dry sodium acrylate, 20 mg phenothiazine, and 19.162 g of PPL produced from ring-opening polymerization of solvent-free BPL in the presence of sodium acrylate at a concentration of 1 mol per 6,000 mol of BPL and phenothiazine at a concentration of 200 ppmw in BPL. The feed stream in the thermolysis process vessel is heated to 90° C. to melt and begin stirring. The thermolysis process vessel is brought under vacuum to an absolute pressure of approximately 90 torr, and the thermolysis process vessel temperature setpoint is set to 165° C. Internal reflux is observed inside the thermolysis process vessel within minutes. The thermolysis process vessel is held at 165° C. for 40 minutes.

The product sample 129-108_Dist HNMR analysis suggests an average acrylic acid content in 129-108_Dist of 99.7%. The balance consists of di-acrylic acid ester and traces of other PPL oligomers where n>2.

The embodiments described herein are not intended to be limited to the aspects shown, but are to be accorded the widest scope consistent with the principles and features disclosed herein.

What is claimed is:

1. A composition for producing a high purity acrylic acid thermolysis product, comprising:
a. polypropiolactone; and
b. one or more active salts for catalyzing thermolysis of said polypropiolactone when said composition undergoes thermolysis at thermolysis conditions.

2. The composition of claim 1, wherein a portion of said active salt is residual from a polymerization reaction producing said polypropiolactone.

3. The composition of claim 1, wherein said composition has a polypropiolactone concentration of at least 95% by weight.

4. The composition of claim 1, wherein said composition has polypropiolactone concentration of at least 98% by weight.

5. The composition of claim 1, wherein said polypropiolactone has a number average molecular weight between 750 g/mol and 10,000 g/mol.

6. The composition of claim 1, wherein said polypropiolactone has a number average molecular weight of greater than 10,000 g/mol.

7. The composition of claim 1, wherein said composition has a concentration of said one or more active salts of at least 0.01 by weight %

8. The composition of claim 1, wherein said composition has a concentration of said one or more active salts of at least 1% by weight.

9. The composition of claim 1, wherein said composition has a concentration of said one or more active salts less than 10% by weight.

10. The composition of claim 1, wherein said composition has one or more active salts chosen from the group including sodium acrylate, potassium acrylate, sodium carbonate, potassium carbonate, and tert-butyl ammonium acrylate.

11. The composition of claim 1, wherein the composition further comprises phenothiazine.

12. A composition for producing a high purity acrylic acid thermolysis product, comprising:
a. polypropiolactone having with a concentration of at least 95% by weight and a number average molecular weight between 750 g/mol and 10,000 g/mol; and
b. one or more active salts with a concentration of at least 0.01% by weight for catalyzing thermolysis of said polypropiolactone when said composition undergoes thermolysis at thermolysis conditions.

13. The composition of claim 12, wherein a portion of said one or more active salts is residual from a polymerization reaction producing said polypropiolactone.

14. The composition of claim 12, wherein said composition has polypropiolactone concentration of at least 98% by weight.

15. The composition of claim 12, wherein said composition has a concentration of said one or more active salts less than 10% by weight.

16. The composition of claim 12, wherein said composition has one or more active salts chosen from the group including sodium acrylate, potassium acrylate, sodium carbonate, potassium carbonate, and tert-butyl ammonium acrylate.

17. A composition for producing a high purity acrylic acid thermolysis product, comprising:
a. polypropiolactone with a concentration of at least 98% by weight and a number average molecular weight greater than 10,000 g/mol; and
b. one or more active salts with a concentration between 0.5% and 1.5% by weight for catalyzing thermolysis of said polypropiolactone when said composition undergoes thermolysis at thermolysis conditions.

18. The composition of claim 17, wherein a portion of said one or more active salts is residual from a polymerization reaction producing said polypropiolactone.

19. The composition of claim 17, wherein said composition has polypropiolactone concentration of at least 98% by weight.

20. The composition of claim 17, wherein said composition has a concentration of said one or more active salts less than 10% by weight.

21. The composition of claim 17, wherein said composition has one or more active salts chosen from the group including sodium acrylate, potassium acrylate, sodium carbonate, potassium carbonate, and tert-butyl ammonium acrylate.

22. The composition of claim 1, wherein said one or more active salts are residual polymerization catalyst from polymerization of said polypropiolactone.

23. The composition of claim 12, wherein said one or more active salts are residual polymerization catalyst from polymerization of said polypropiolactone.

24. The composition of claim 17, wherein said one or more active salts are residual polymerization catalyst from polymerization of said polypropiolactone.
